(19) United States
(12) Patent Application Publication
(10) Pub. No.: US 2015/0174371 A1
(43) Pub. Date: Jun. 25, 2015

(54) SYSTEM FOR BYPASSING VASCULAR OCCLUSION HAVING PUNCTURING MECHANISM AND METHOD

(71) Applicant: Cook Medical Technologies LLC, Bloomington, IN (US)

(72) Inventors: Jeremy Schaeffer, Bloomington, IN (US); David Christian Lentz, Bloomington, IN (US); Logan Cage, Bloomington, IN (US); James Elsesser, Bloomington, IN (US); Richard Alley, Bloomington, IN (US); Charles Tomasek, Rochester, MN (US)

(21) Appl. No.: 14/526,935

(22) Filed: Oct. 29, 2014

Related U.S. Application Data
(60) Provisional application No. 61/920,121, filed on Dec. 23, 2013.

(51) Int. Cl. A61M 25/01; A61B 17/3207; A61B 17/22; A61B 17/32

(52) U.S. Cl. CPC A61M 25/0194; A61B 17/32; A61B 17/320783; A61B 17/3207; A61B 17/22; A61B 2017/320056; A61B 2017/320791; A61B 2017/22001; A61B 2017/22095; A61M 2025/0197

(57) ABSTRACT

A system for bypassing a vascular occlusion includes a catheter, and a puncturing mechanism within the catheter having a sleeve and a puncture wire positioned within the sleeve. The puncture wire is elastically deformable between an access configuration, and a deployed configuration. The puncture wire has a shape memory bias and is retracted within the sleeve and held by the sleeve in opposition to the bias in the access configuration.

### TECHNICAL FIELD

The present disclosure relates generally to bypassing a vascular occlusion in a patient, and more particularly to an occlusion bypassing system having a puncturing mechanism with a sleeve and elastically deformed puncture wire within the sleeve having a piercing tip.

### BACKGROUND

Thrombosis is the formation of a thrombus, or blood clot, within the vascular system of a patient. When attached to vessel walls, blood clots and other substances such as plaque or fat may reduce or block blood flow. Chronic total occlusion (CTO) is a complete blockage within the vascular system, commonly formed by fibrous calcified material. Blockage of blood flow by a CTO may prevent critical oxygen from reaching certain tissues. Regardless of the particular location within the vascular system, a CTO, if left untreated, may cause serious damage and, in some cases, may be life threatening.

A wide variety of techniques are available for treating blockage in the vasculature. Some percutaneous techniques include the use of pharmacological agents, to help dissolve the blockage material. Other percutaneous techniques may include the use of a wire guide and/or catheter to cross an occlusion and recanalize the vessel. Crossing a CTO using a wire guide and/or catheter may be difficult and, oftentimes, impossible, due to the hardness of the blockage material. During these recanalization procedures, it is common for the wire guide to be inadvertently advanced into the subintimal space of the vessel wall. Once the wire guide has entered the subintimal space, either inadvertently or intentionally, it may be possible to create a new lumen through the subintimal space that bypasses the clot, such as by performing an angioplasty procedure. However, it is often difficult to redirect the wire guide back into the true lumen of the vessel at a distal location relative to the occlusion.

An exemplary lumen re-entry device is described in U.S. Patent Application Publication No. 2007/0219464 to Davis et al. Specifically, the Davis et al. reference teaches a steerable guide wire having a sharpened re-entry tip. The guide wire comprises a hypotube having a helical coil attached to and extending from a distal end of the hypotube. A retaining ribbon is connected to the distal end of the hypotube and is also connected to the sharpened re-entry tip. A deflection member is slidably disposed within the hypotube and has a distal end connected to the sharpened re-entry tip such that distal movement of the deflection member deflects the sharpened re-entry tip in one direction, while proximal movement of the deflection member deflects the sharpened re-entry tip in an opposite direction. While the lumen re-entry device of Davis et al. might offer certain advantages, there is always room for improvement.

### SUMMARY OF THE DISCLOSURE

In one aspect, a system for bypassing a vascular occlusion in a patient includes a catheter having an elongate catheter body having formed therein a longitudinally extending wire guide lumen and a longitudinally extending second lumen. The system further includes a puncturing mechanism having a sleeve slidable within the second lumen, and a puncture wire positioned within the sleeve and including a proximal segment defining a longitudinal axis and a distal segment having a piercing tip. The puncture wire is elastically deformable between an access configuration where the distal segment is straightened and the piercing tip is positioned closer to the longitudinal axis, and a deployed configuration where the distal segment is curved and the piercing tip is positioned further from the longitudinal axis. The puncture wire further has a shape memory bias and is at a retracted position where the sleeve holds the puncture wire in opposition to the shape memory bias in the access configuration. The puncture wire is slidable to an advanced position where the distal segment projects out of the sleeve and the shape memory bias positions the puncture wire in the deployed configuration.

In another aspect, a puncturing mechanism for a vascular occlusion bypassing system includes a sleeve having a proximal end, a distal end, a cylindrical outer surface, and a cylindrical inner surface coaxial with the cylindrical outer surface and defining a longitudinally extending central lumen opening at each of the proximal and distal ends. The puncturing mechanism further includes a puncture wire positioned within the central lumen and including a proximal segment defining a longitudinal axis and a distal segment having a piercing tip, for forming an opening between a subintimal space and a vascular lumen in a patient. The puncture wire has a shape memory bias and is elastically deformable between an access configuration where the distal segment is straightened and the piercing tip is positioned closer to the longitudinal axis, and a deployed configuration where the distal segment is curved and the piercing tip is positioned further from the longitudinal axis. The puncture wire is at a retracted position where the sleeve contacts the distal segment and holds the puncture wire in opposition to the shape memory bias in the access configuration, and is slidable within the sleeve to an advanced position where the distal segment projects out of the sleeve and the shape memory bias positions the puncture wire in the deployed configuration.

In still another aspect, a method of bypassing a vascular occlusion in a patient includes advancing a puncturing mechanism having a sleeve and a puncture wire within the sleeve through a subintimal space about a vascular occlusion in a patient, and holding the puncture wire during the advancement in an access configuration where a distal segment of the puncture wire is straightened by the sleeve in opposition to a shape memory bias of the puncture wire. The method further includes sliding the puncture wire through the sleeve such that the distal segment projects out of the sleeve and is curved via the shape memory bias to position the puncture wire in a deployed configuration. The method further includes forming an opening between the subintimal space and a vascular lumen in the patient via a piercing tip of the distal segment in the deployed configuration of the puncture wire, and advancing the sleeve through the opening such that the sleeve forms a conduit extending through the subintimal space from a first side of the vascular occlusion to a second side.

### BRIEF DESCRIPTION OF THE DRAWINGS

FIG. 1 is a partially sectioned side diagrammatic view, including a detailed enlargement, of a system according to one embodiment;

FIG. 2 is a partially sectioned side diagrammatic view of a portion of a puncturing mechanism for use in the system of FIG. 1;

FIG. 3 is a side diagrammatic view at one stage of a procedure, according to one embodiment;

FIG. 4 is a side diagrammatic view at another stage of the procedure; and

FIG. 5 is a side diagrammatic view at yet another stage of the procedure.

### DETAILED DESCRIPTION

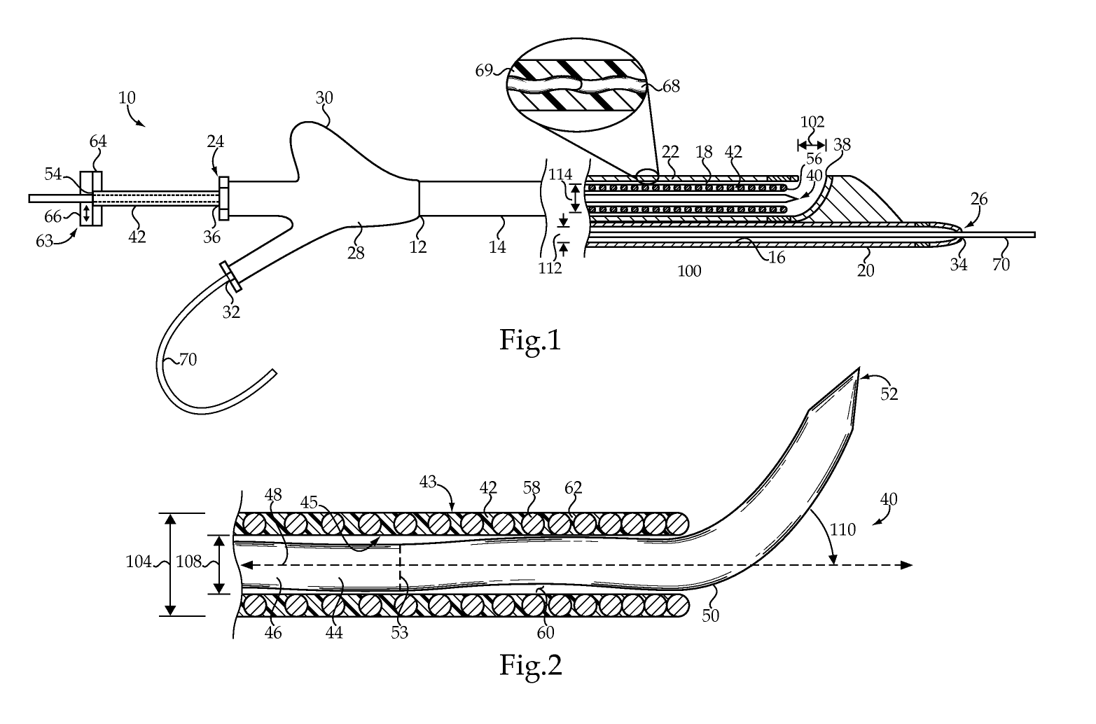

Referring to FIG. 1, there is shown a system 10 for bypassing a vascular occlusion in a patient. System 10 includes a catheter 12 with an elongate catheter body 14 having formed therein a longitudinally extending wire guide lumen 16, and a longitudinally extending second lumen 18. In a practical implementation strategy, catheter body 14 is formed from a first tubular body 20 attached to a second tubular body 22. Catheter body 14 has a proximal catheter body end 24, and a distal catheter body end 26 which is formed by second tubular body 22. Tubular bodies 20 and 22 may be attached to one another via a suitable adhesive, melt forming, or via any other suitable strategy. Embodiments are nevertheless contemplated where catheter body 14 is formed from a single extrusion taking the place of first and second tubular bodies 20 and 22. Catheter 12 further includes a manifold 28 having a projecting fin 30 or the like which enables a clinician to hold catheter 12, and use fin 30 for rotating catheter 12 to manipulate the same within a body lumen in a patient. An inlet 32 to wire guide lumen 16 is formed at proximal catheter body end 24, as is an inlet 36 to second lumen 18. In the FIG. 1 illustration, a wire guide 70 extends into inlet 32, longitudinally through wire guide lumen 16, and out of an outlet 34 at distal catheter body end 34. An outlet 38 of second lumen 18 is formed by second tubular body 22, and is positioned proximally of outlet 34 in the illustrated embodiment. Outlet 38 may have the general form of a side port, formed by a curved shape of tubular body 22. Radiopaque marker bands or the like (not numbered) may be attached to first and second tubular bodies 20 and 22. System 10 further includes a puncturing mechanism 40 having a sleeve 42 slidable within second lumen 18, and a puncture wire 44 positioned within sleeve 42. As will be further apparent from the following description, system 10 may be uniquely configured by way of the assembly of catheter 12 and puncturing mechanism 40 for bypassing an occlusion such as a chronic total occlusion (CTO) in a patient.

Referring also now to FIG. 2, puncture wire 44 includes a proximal wire segment 46 defining a longitudinal axis 48 and a distal wire segment 50 having a piercing tip 52. Tip 52 may be conical, beveled, faceted or some combination of these or other sharpening techniques. A tip of proximal segment 46 would typically not be configured for piercing. Puncture wire 44 may be elastically deformable between an access configuration and a deployed configuration. In the access configuration, distal segment 50 is straightened and piercing tip 52 is positioned closer to longitudinal axis 48. In the deployed configuration distal segment 50 is curved and piercing tip 52 is positioned further from longitudinal axis 48. In FIG. 2, distal segment 50 defines an angle 110 with longitudinal axis 48. Angle 110 may be from about 10° to about 90°. Also shown in FIG. 2 is an imaginary line 53 between distal segment 50 and proximal segment 46 of puncture wire 44. In a practical implementation strategy puncture wire 44 has a shape memory bias. Puncture wire 44 will tend to assume a rest shape other than a linear shape when no external force opposing the shape memory bias is applied. In the illustrated embodiment, distal segment 50 may be considered that part of puncture wire 44 positioned distally, on the right side, of line 53, whereas proximal segment 46 is the part of puncture wire 44 on the left side of line 53. Were puncture wire 44 to be removed from sleeve 42 altogether, proximal segment 46 might have a generally linear shape, whereas all of distal segment 50 might have a curved shape. Accordingly, at the state shown in FIG. 2 puncture wire 44 could be advanced further out of sleeve 42 to curve more steeply still, but can nevertheless be considered to be in a deployed configuration. It can thus be appreciated that contact between puncture wire 44 and an inner sleeve surface 45 causes the straightening of puncture wire 44, and in particular distal segment 50, when puncture wire 44 is at a retracted position within sleeve 42 as in FIG. 1. Another way to understand these principles is that while puncture wire 44 is understood to have a shape memory bias, the shape memory bias of puncture wire 44 can further be understood as a subordinate shape memory bias while sleeve 42 can be understood to have a dominant shape memory bias. Sleeve 42 further has an outer sleeve surface 43 which may be cylindrical, and concentric with inner sleeve surface 45, also typically cylindrical.

As noted above, sleeve 42 is slidable within second lumen 18, meaning that the assembly of sleeve 42 and puncture wire 44 forming puncturing mechanism 40 can be slid out of second lumen 18 as an assembly. Alternatively, puncture wire 44 can be slid out of sleeve 42 without extending sleeve 42 out of second lumen 18. Embodiments are contemplated where a clinician can move puncture wire and sleeve 42 together out of second lumen 18, then advance puncture wire out of sleeve 42 to puncture tissue, and then further advance sleeve 42 to re-sheath puncture wire 44. Puncture wire 44 might then be withdrawn while holding sleeve 42 stationary, and swapped with a softer wire guide that can be used for the guiding and placement of other interventional devices such as a balloon for restoring blood flow through a vessel. These and other capabilities of the present system will be understood by way of the example implementations discussed below.

The ability to advance sleeve 42 and puncture wire 44 together, or independently, is facilitated at least in part through the use of a locking mechanism 63 coupled to sleeve 42 and puncture wire 44. In a practical implementation strategy, locking mechanism 63 has an unlocked configuration where sleeve 42 and puncture wire 44 are slidable relative one another, and a locked configuration where sleeve 42 and puncture wire 44 are locked against sliding relative one another. Mechanism 63 may include a base 64 attached, for example, to sleeve 42, and a sliding tab 66 or the like slidably coupled to base 64. In one example strategy, tab 66 is slidable between a first position at which it does not contact puncture wire 44 to a second position at which tab 66 contacts and frictionally engages with puncture wire 44, allowing a clinician to alternately unlock and lock mechanism 63 as desired. In other instances, mechanism 63 might include a lever, a button, or any other suitable device that can be selectively actuated to alternate puncture wire 44 and sleeve 42 between a state where they can be slid relative one another and a state where they are locked against sliding relative one another. Base 64 may be attached to a proximal sleeve end 54, and puncture wire 44 may be slidable out of distal sleeve end 56.

In a further practical implementation strategy, sleeve 42 may include a skeleton 58 extending circumferentially around a central lumen 60. Puncture wire 44 may be slidable within central lumen 60 between its refracted position where sleeve 42 holds puncture wire 44 in opposition to its shape memory bias in the access configuration, and its advanced position where distal segment 50 projects out of sleeve 42 and the shape memory bias positions puncture wire 44 in the deployed configuration. In the illustrated embodiment, skeleton 58 has the form of a helical wire, forming a plurality of turns about longitudinal axis 48 of puncture wire 44 when puncture wire 44 is positioned therein. The turns formed by skeleton 58 may be relatively denser at the proximal and distal sleeve ends 54 and 56, and relatively less dense between ends 54 and 56. Sleeve 42 may further include a tubular jacket 62 formed of a polymeric material, such as a fluoropolymer material, attached to helical wire 58 which may be metallic. Tubular jacket 62 may encase helical wire 58, such that sleeve 42 has both sufficient stiffness to oppose the shape memory bias of puncture wire 44 and straighten puncture wire 44 when refracted, and also sufficient column strength to enable puncturing mechanism 44 to be pushed out of second lumen 18, or to enable catheter 12 to be retracted while puncturing mechanism 40 is held at a fixed position. Jacket 62 and skeleton/wire 58 may thus work together to inhibit curving of puncture wire 44 and enable manipulation and adjustment of puncturing mechanism 40 within a patient.

It may further be noted from FIG. 1 that first tubular body 20 defines wire guide lumen 16 and second tubular body 22 defines second lumen 18, with each of first and second tubular bodies 20 and 22 extending between proximal and distal body ends 24 and 26 and distal end 26 being formed by first tubular body 20. Tubular bodies 20 and 22 may include a braid, and in the detailed enlargement of FIG. 1 it can be seen that second tubular body 22 includes a metallic braid 68 attached to or encased in polymeric tube material 69. Braid 68 facilitates torquing of catheter 12, and could extend around both of tubular bodies 20 and 22 or only tubular body 22 as in the illustrated embodiment. It may further be noted from FIG. 1 that tubular body 20 is smaller in diameter than tubular body 22. In a practical implementation strategy lumen 16 is no larger than needed to accommodate a wire guide from about 0.014 inches to about 0.016 inches in diameter, therefore an inner diameter dimension 112 of tubular body 20 may be about 0.016 inches or only slightly larger, and could be only slightly larger than about 0.014 inches. An inner diameter dimension 114 of second lumen 18 may be about 0.035 inches, and a diameter 102 of outlet 38 may be about 0.039 inches. An outer diameter dimension 104 of sleeve 42 may be about 0.035 inches, and an inner diameter dimension 108 of sleeve 42 may be about 0.018 inches. A length of sleeve 42 from proximal sleeve end 54 to distal sleeve end 56 might be about 170 cm, whereas a length of puncture wire 44 from tip to tip may be about 5 cm to about 15 cm greater than the length of sleeve 42. Puncture wire 44 itself might be from about 0.014 inches to about 0.019 inches in diameter, and formed from a suitable metallic alloy such as nitinol or 304V stainless steel. Catheter 12 may be overall from about 4 French to about 5 French diameter. As used herein, the term “about” should be understood in the context of conventional rounding to a consistent number of significant digits. Accordingly, “about 0.016” means from 0.0155 to 0.0164, and so on.

### INDUSTRIAL APPLICABILITY

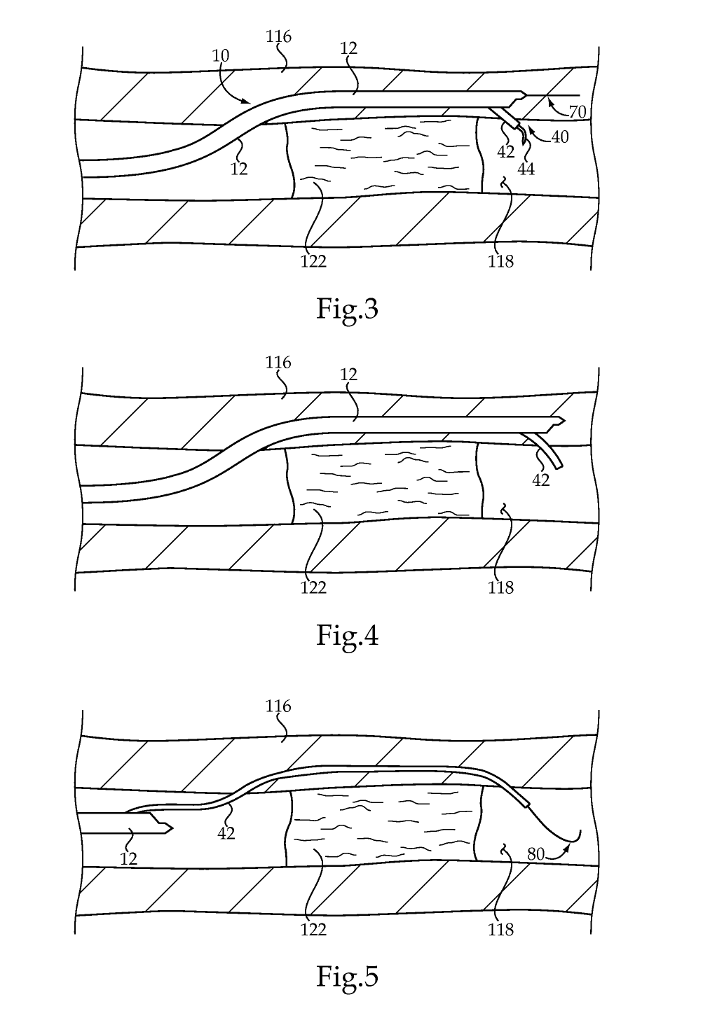

Referring to the drawings generally, but in particular now to FIG. 3, there is shown system 10 at a state where catheter 12 has been advanced through a vascular lumen 118 in a vessel 116 such as a vein or artery, in particular via sliding over wire guide 70 such that catheter 10 extends through a subintimal space 116 past an occlusion 122 in a vessel 100. During the advancement of catheter 12, puncturing mechanism 40 is positioned therein and sleeve 42 holds distal segment 50 of puncture wire 44 in the access configuration such that distal segment 50 is straightened by sleeve 42 in opposition to the shape memory bias of puncture wire 44. With system 10 positioned thusly, locking mechanism 63 may be unlocked and puncture wire 44 slid through sleeve 42 such that distal segment 50 projects out of sleeve 42 and is curved via the shape memory bias to position puncture wire 44 in a deployed configuration, much as it might appear in FIG. 3. During the sliding of the puncture wire out of sleeve 42, or subsequently, puncture wire 44 may be used to form an opening between subintimal space 116 and a vascular lumen 118 via piercing tip 52. During the forming of the opening, or subsequently, sleeve 42 may be advanced through the opening such that sleeve 42 forms a conduit extending through subintimal space 16 from a first side of occlusion 122 to a second side, as shown in FIG. 4.

With sleeve 42 extending through subintimal space 116 from the first to second sides of vascular occlusion 122, puncture wire 44 may be withdrawn from sleeve 42 and withdrawn from the patient altogether. Catheter 12 may also be withdrawn, potentially such that no part of system 10 is positioned in the patient apart from sleeve 42. Typically but not necessarily prior to withdrawing catheter 12, a substitute wire guide 80 as shown in FIG. 5 may be advanced through sleeve 42 such that wire guide 80 also extends through subintimal space 116 to form a bypass around vascular occlusion 122.

Those skilled in the art will appreciate that the techniques depicted in FIGS. 3, 4 and 5 and the accompanying description demonstrate that sleeve 42 will be capable of acting much like a miniature catheter bypassing the occlusion, which can be used for purposes such as the delivery of interventional tools, injection of fluids such as contrast agent, or for still other purposes such as the positioning of wire guide 80 to enable navigation deeper into the vasculature. From the state depicted in FIG. 5, sleeve 42 might be withdrawn to leave only wire guide 80 bypassing occlusion 122, from which point a clinician might position a balloon catheter or a stent through subintimal space 116.

The present description is for illustrative purposes only, and should not be construed to narrow the breadth of the present disclosure in any way. Thus, those skilled in the art will appreciate that various modifications might be made to the presently disclosed embodiments without departing from the full and fair scope and spirit of the present disclosure. Other aspects, features and advantages will be apparent upon an examination of the attached drawings and appended claims.

What is claimed is:

1. A system for bypassing a vascular occlusion in a patient comprising:
a catheter including an elongate catheter body having formed therein a longitudinally extending wire guide lumen and a longitudinally extending second lumen;
a puncturing mechanism including a sleeve slidable within the second lumen, and a puncture wire positioned within the sleeve and including a proximal segment defining a longitudinal axis and a distal segment having a piercing tip;
the puncture wire being elastically deformable between an access configuration where the distal segment is straightened and the piercing tip is positioned closer to the longitudinal axis, and a deployed configuration where the distal segment is curved and the piercing tip is positioned further from the longitudinal axis; and
the puncture wire further having a shape memory bias and being at a retracted position where the sleeve holds the puncture wire in opposition to the shape memory bias in the access configuration, and being slidable to an advanced position where the distal segment projects out of the sleeve and the shape memory bias positions the puncture wire in the deployed configuration.

2. The system of claim 1 wherein the shape memory bias is a subordinate shape memory bias, and the sleeve includes a dominant shape memory bias.

3. The system of claim 1 wherein the sleeve includes a skeleton extending circumferentially around a central lumen, and the puncture wire is slidable within the central lumen between the retracted and advanced positions.

4. The system of claim 3 wherein the skeleton includes a helical wire.

5. The system of claim 4 wherein the sleeve further includes a tubular jacket formed of a polymeric material and attached to the helical wire.

6. The system of claim 4 wherein the helical wire is encased by the tubular jacket.

7. The system of claim 1 further comprising a locking mechanism having an unlocked configuration where the sleeve and puncture wire are slidable relative one another, and a locked configuration where the sleeve and puncture wire are locked against sliding relative one another.

8. The system of claim 1 wherein the elongate catheter body includes a proximal end and a distal end, and a first and a second tubular body each extending between the proximal and distal ends and defining the wire guide lumen and the second lumen, respectively.

9. The system of claim 8 wherein the wire guide lumen has a smaller inner diameter dimension, and the second lumen has a larger inner diameter dimension.

10. The system of claim 9 wherein the second lumen has a second lumen outlet, and the wire guide lumen has a wire guide lumen outlet located distal to the second lumen outlet.

11. A puncturing mechanism for a vascular occlusion bypassing system comprising:
a sleeve including a proximal end, a distal end, a cylindrical outer surface, and a cylindrical inner surface coaxial with the cylindrical outer surface and defining a longitudinally extending central lumen opening at each of the proximal and distal ends;
a puncture wire positioned within the central lumen and including a proximal segment defining a longitudinal axis and a distal segment having a piercing tip, for forming an opening between a subintimal space and a vascular lumen in a patient;
the puncture wire having a shape memory bias and being elastically deformable between an access configuration where the distal segment is straightened and the piercing tip is positioned closer to the longitudinal axis, and a deployed configuration where the distal segment is curved and the piercing tip is positioned further from the longitudinal axis; and
the puncture wire being at a retracted position where the sleeve contacts the distal segment and holds the puncture wire in opposition to the shape memory bias in the access configuration, and being slidable within the sleeve to an advanced position where the distal segment projects out of the sleeve and the shape memory bias positions the puncture wire in the deployed configuration.

12. The puncturing mechanism of claim 11 further comprising a locking mechanism having an unlocked configuration where the sleeve and puncture wire are slidable relative one another, and a locked configuration where the sleeve and puncture wire are locked against sliding relative one another.

13. The puncturing mechanism of claim 11 wherein the sleeve includes a skeleton extending circumferentially around a central lumen, and the puncture wire is slidable within the central lumen between the retracted and advanced positions.

14. The puncturing mechanism of claim 12 wherein the skeleton includes a helical wire, and wherein the sleeve further includes a tubular jacket formed of a polymeric material and encasing the helical wire.

16. A method of bypassing a vascular occlusion in a patient comprising the steps of:
advancing a puncturing mechanism having a sleeve and a puncture wire within the sleeve through a subintimal space about a vascular occlusion in a patient;
holding the puncture wire during the advancement in an access configuration where a distal segment of the puncture wire is straightened by the sleeve in opposition to a shape memory bias of the puncture wire;
sliding the puncture wire through the sleeve such that the distal segment projects out of the sleeve and is curved via the shape memory bias to position the puncture wire in a deployed configuration;
forming an opening between the subintimal space and a vascular lumen in the patient via a piercing tip of the distal segment in the deployed configuration of the puncture wire; and
advancing the sleeve through the opening such that the sleeve forms a conduit extending through the subintimal space from a first side of the vascular occlusion to a second side.

17. The method of claim 16 further comprising a step of unlocking a locking mechanism inhibiting sliding of the puncture wire relative the sleeve, prior to the step of sliding the puncture wire.

18. The method of claim 17 further comprising a step of relocking the locking mechanism prior to the step of advancing the sleeve.

19. The method of claim 16 further comprising the steps of advancing the puncturing mechanism out of a lumen in a catheter prior to the step of forming an opening, and withdrawing the catheter from the patient via sliding the catheter over the sleeve while the conduit is formed by the sleeve.

20. The method of claim 16 wherein the step of holding further includes inhibiting curving of the puncture wire via a helical wire skeleton and attached tubular jacket of the sleeve.
